(19) United States
(12) Patent Application Publication
(10) Pub. No.: US 2010/0086507 A1
(43) Pub. Date: Apr. 8, 2010

(54) MAKE-UP METHOD AND SET FOR CARRYING OUT SAID METHOD

(76) Inventor: Jean-Louis Gueret, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/526,026

(22) PCT Filed: Feb. 6, 2008
(86) PCT No.: PCT/FR2008/050181

§ 371 Date: Dec. 7, 2009

(30) Foreign Application Priority Data
Feb. 6, 2007 (FR) 0753096

(51) Int. Cl. A61K 8/00; A61Q 1/10

(52) U.S. Cl. 424/70.7

(57) ABSTRACT

The present invention relates to a method for making-up the eyelashes, comprising the steps made up of:
1) cold application of a composition (P) to the eyelashes using a non-heating applicator, this composition tending to set by drying;
2) before the composition dries completely, bringing a finishing device comprising a hot surface that is subjected to vibrations into contact with the eyelashes thus coated with the composition, so as to accelerate the drying of the composition.

The present invention relates to making-up the eyelashes.

Curling of the eyelashes is a feature very often desired by mascara users.

It is relatively difficult to obtain a completely satisfactory curling of the eyelashes using a brush or a comb impregnated with a composition containing one or more solvents which by evaporating therefrom cause the mascara to set, since bringing the applicator into contact with the eyelashes in order to curl them tends to introduce fresh product onto the eyelashes. This may slow down the evaporation of the solvent or solvents from the deposition of product previously made and/or re-dissolve it, so that the mascara does not necessarily set the eyelashes in the desired configuration.

It has also been proposed to use compositions that have a high-temperature threading nature, the application being carried out with a device comprising a hot surface raised to a high enough temperature to make the composition melt.

The application of a mascara having a high-temperature threading nature however proves quite tricky, a large number of parameters possibly rendering the operation difficult.

Application EP 1 543 741 A2 describes a device comprising a heating member and projecting elements arranged so as to selectively make it possible to either use the projecting elements to separate the eyelashes without contact of the latter with the heating member, or to curl the eyelashes by subjecting them to the heat released by the heating member.

It has been proposed, in the publication WO 2006/090343 in the name of the Applicant, to use a vibrating finishing element in order to perfect a making-up, by bringing this element into contact with keratin materials that are already made-up. It is also proposed in this document to make an applicator vibrate that applies a hot composition.

U.S. Pat. No. 2,630,516 discloses an eyelash curling iron equipped on the outside with a sheath of a fibrous material, preferably a chamois skin, with a view to retaining excess mascara. In that patent, heat is used to set the mascara.

EP 1 468 627 A1 describes a heating applicator for applying a viscous composition having a particular temperature profile. The composition is deposited on the applicator by immersing the latter in a flask containing the composition, equipped with a wiping member.

There is a need to facilitate and improve the eyelash curling operation. The invention aims to meet this need in a manner that is simple and easy to implement by the greatest number of users.

According to one of its aspects, one subject of the invention is a method for making-up the eyelashes, comprising the steps made up of:

1) cold application of a composition comprising one or more volatile solvents to the eyelashes using a non-heating applicator;

2) before the composition dries completely, bringing a finishing device comprising a hot surface that is subjected to vibrations into contact with the eyelashes thus coated with the composition.

The expression “cold” should be understood to mean that the composition is applied at ambient temperature.

The application of the composition to the eyelashes may be carried out in step 1) using a brush or a comb, in a conventional manner.

The application of the composition in this step 1) may be carried out easily, since it is enough for the user to coat the eyelashes with the composition, without seeking to give them maximum curvature, the step of curling to the desired curvature being carried out afterwards. The vibrations may prevent the eyelashes from adhering too much to the hot surface.

By virtue of the invention, the heat and the vibrations of the finishing device accelerate the evaporation of the solvent or solvents contained in the composition and it is thus possible to obtain, relatively rapidly, the drying of the composition deposited on the eyelashes, while using the finishing device to curl the eyelashes, by lifting them for example.

Furthermore, since the finishing device is different from the applicator that has been used to deposit the composition on the eyelashes, it is possible to prevent the introduction of fresh product during the finishing step, the consequence of which would be to slow down the evaporation of the solvent or solvents.

The hot surface used above is advantageously a smooth and/or non-porous surface, which facilitates the cleaning thereof.

The hot surface may also comprise striations, teeth and/or grooves.

The finishing device may also comprise striations, teeth and/or grooves elsewhere than at the hot surface, for example on the opposite side from the latter.

The hot surface may be of tapered shape, which may facilitate its engagement between the eyelashes, if necessary, in order to separate them.

The hot surface may have an axisymmetric shape, especially a symmetrical axisymmetric shape.

The hot surface may have a temperature less than or equal to 90° C., better still between 35 and 85° C., even better still 45° and 80° C., and for example a homogeneous temperature.

The hot surface may be defined by one portion at least of a finger-shaped heating support. The end of this finger may be rounded, in order to avoid risk of injury to the user. The heating support may be free of teeth or of bristles that are oriented transversely.

The hot surface may be subjected to vibrations oriented parallel or transverse to its longitudinal axis.

The amplitude of the vibrations of the heating support is preferably less than or equal to 5 mm, better still 3 mm, microvibrations being preferable.

The frequency of the vibrations is preferably between 1 and 500 Hz.

The hot surface may be vibrated by means of a vibrating source integrated into the finishing device.

As a variant, the hot surface may be vibrated by means of a vibrating source removably added to the finishing device or by means of a vibrating source borne by a finger of the user and brought into contact with the finishing device.

The hot surface may be free of composition before its first contact with the keratin fibers. This may improve the heat transfer and avoid depositing composition residues which would have dried onto the eyelashes.

The method may comprise, where appropriate, the cleaning of the hot surface during the use thereof, for example by means of a piece of a non-woven fabric or of a textile, of a sponge or of a paper. The cleaning may be carried out while the hot surface is at an operating temperature or in the absence of heating.

Another subject of the invention, according to another of its aspects, is an assembly comprising, within one and the same packaging:
a finishing device comprising a heating surface;
a vibrating source that enables the heating surface to be vibrated, this vibrating source being integrated into the finishing device or possibly being removably fastened to this device or possibly being brought into contact with this device; and
the composition to be applied to the eyelashes.

The packaging is, for example, a blister pack, a box or a sachet.

Yet another subject of the invention is the finishing device as is, independently of the composition. This finishing device may or may not incorporate the vibrating source.

The finishing device may comprise a finger-shaped heating support, which at least partially defines the heating surface.

The heating support may comprise an unheated portion at its free end. This may make it possible to use the heating support to separate the eyelashes without heating them, by using the tip.

The hot surface may be defined at least partially, or even completely, by a metal, a glass, a ceramic or an elastomer.

The heating support may comprise an envelope having a housing in which at least one heating resistor is placed, this housing opening for example at one end of the envelope, so as to allow the heating resistor to be inserted into the envelope, after the manufacture of the envelope. This housing may be central, so as to enable, if this is desired, a homogeneous temperature to be obtained around the longitudinal axis of the heating support.

As a variant, the heating support comprises an envelope which is overmolded onto the heating resistor.

The envelope may be made from a material that is a good conductor of heat, for example a metal or a thermoplastic or crosslinkable material filled with metallic particles or having a good thermal conductivity.

The finishing device may comprise at least one switch to enable the user to start the heating of the heating support. This switch is for example a contactor, for example a push button, that starts the heating of the heating support when pushed in by the user, the heating of the heating support stopping for example as soon as the contactor is released by the user. This may enable the user to create heating pulses that are relatively short and well controlled over time, which may facilitate the finishing operation.

The finishing device may also comprise a switch for controlling the vibrating source. This may enable the user to carry out the finishing operation by selectively using either vibrations alone, or heating alone, or both.

In one implementation variant of the invention, the finishing device comprises a switch which may take up a position in which, even once released by the user, the heating support and/or the vibrating source remain switched on, where appropriate for a predefined duration so as to save the electrical source.

The finishing device may also comprise a light indicator that gives the user information for example about whether the heating support is switched on or off and/or about the temperature of this heating support.

The light indicator is for example a light that lights up when the heating support is electrically switched on and which changes color, flashes or stops flashing when the suitable finishing temperature is reached.

The finishing device may also comprise a thermochromic indicator, for example that changes color when a suitable operating temperature is reached.

The finishing device may comprise a casing that houses an electrical source, for example at least one battery or storage cell. The finishing device may also be powered by the mains, for example through a step-down transformer. The use of a 9V battery for powering the heating support may increase the current intensity in the resistor and enable a faster rise in temperature, which may facilitate the making-up operation.

The electrical source and the heating support are, for example, chosen so that the heating support can reach a surface temperature between 45° C. and 95° C., better still 45° C. and 90° C., even better still of at least 60° C., in less than 120 s, better still 30 s, starting from an initial temperature of 25° C. in an enclosed environment at 25° C.

The heating support may be made from a material having a finish and/or a surface tension so that the composition does not adhere, at 25° C., to the heating support. The heating support may be made from PTFE (Teflon®) or have a coating of PTFE or of a similar material.

In one implementation example of the invention, the heating support is fastened relative to a gripping portion of the finishing device.

In another implementation example of the invention, the heating support is movable relative to a gripping portion of the finishing device.

The heating support is for example translatably movable. This may make it possible to reduce the bulkiness of the finishing device when not being used.

The heating support may also be rotatably movable, for example about an axis of rotation perpendicular to its longitudinal axis, about itself or about any axis that does not coincide with its longitudinal axis.

The heating support may have any shape suitable for the desired making-up operation.

The heating support may have a longitudinal axis which is or is not rectilinear. A curvilinear longitudinal axis may, for example, further improve the ergonomics.

The longitudinal axis of the heating support may coincide with or be parallel to the longitudinal axis of a gripping portion of the finishing device, this gripping portion for example coinciding with a casing of the finishing device.

The longitudinal axis of the support may also be rectilinear and make an angle with the longitudinal axis of the gripping portion of the finishing device.

The support may comprise a heating resistor placed inside an envelope which may have a substantially constant thickness, in order, for example, to more readily obtain a relatively homogeneous temperature over the entire length of the heating support. Where appropriate, the envelope may have a wall thickness that varies longitudinally as a function of a desired temperature profile.

The application device may be arranged so that the temperature of the heating support is, during the operation of the heating support, between 45° C. and 90° C., better still between 50 and 70° C., over at least one portion of its length and over this portion all around the longitudinal axis.

The device may comprise a means for controlling the temperature of the heating support, for example a thermostat or an electronic circuit comprising a temperature probe.

The invention is most particularly suitable for the compositions disclosed in publications EP 1 752 136 A1, EP 1 832 277 A1, EP 0 832 637 A1 and U.S. Pat. No. 6,274,131, the contents of which are incorporated by reference.

The invention is especially suitable for compositions containing a relatively high proportion of water, for example at least 30% water.

The composition may comprise a polymer latex and/or a film-forming polymer.

The composition may also contain ingredients commonly used in cosmetics, such as vitamins, trace elements, softeners, sequestrants, fragrances, oils, silicones, cohesive agents and also alkalinizing or acidifying agents customarily used in the cosmetics field, fillers, pigments, emollients, customarily used in amounts between 1 and 10%; preservatives.

A—Examples of Compositions

In one implementation example of the invention, the composition comprises a mixture of at least one hard wax and of at least one film-forming polymer.

The composition may for example comprise:

i. a waxes-in-water emulsion of at least one wax I, or a mixture of waxes I, having a needle penetration ranging from 1 to 7.5 and a melting point ranging from 70° C. to 110° C., said wax I being at least one wax (Ia, Ib) having a melting point ranging from 77° C. to 110° C., said wax I, or mixtures thereof, being present in a content of at least 10% by weight relative to the total weight of the composition, the wax, or mixture thereof, being in the form of particles having a size at least greater than or equal to 1 μm; and

ii. at least 0.1% by weight, relative to the total weight of the composition, of a polymer system containing a film-forming polymer, said polymer system being capable of forming a film that produces, at a concentration of 7% in water, a greater than 1% shrinkage of isolated stratum corneum at 30° C. and under a relative humidity of 40%.

The composition may comprise:

i. a waxes-in-water emulsion of at least one mixture of waxes I, referred to as hard waxes, having a needle penetration ranging from 1 to 7.5 and a melting point ranging from 70° C. to 110° C., said mixture of waxes I containing at least one first wax (Ia) having a melting point greater than or equal to 77° C. and less than 83° C., a second wax (Ib) having a melting point ranging from 83° C. to 110° C., and a third wax (Ic) having a melting point greater than or equal to 70° C. and less than 77° C., said mixture of waxes I being present in a content of at least 10% by weight relative to the total weight of the composition, said mixture of waxes I being in the form of particles having a size at least greater than or equal to 1 μm; and

ii. 0.1% by weight, relative to the total weight of the composition, of a polymer system containing a film-forming polymer, said polymer system being capable of forming a film that produces, at a concentration of 7% in water, a greater than 1% shrinkage of isolated stratum corneum at 30° C. and under a relative humidity of 40%.

The waxes may be in the form of particles preferably having a size greater than or equal to 1.5 μm, especially ranging from 1.5 to 10 μm, and better still ranging from 1.5 μm to 3.5 μm.

The term “hard wax” (or wax (I)) is understood to mean a wax having a melting point ranging from 70° C. to 110° C. and a needle penetration ranging from 1 to 7.5. The needle penetration of waxes is determined according to French standard NF T 60-123 or US standard ASTM D 1321, at the temperature of 25° C. According to these standards, the needle penetration is the measurement of the depth, expressed in tenths of a millimeter, to which a standardized needle weighing 2.5 g, located in a movable element weighing 97.5 g and placed on the wax to be tested, for 5 seconds, penetrates into the wax.

The waxes I above may be chosen from waxes of animal origin, waxes of plant origin, waxes of mineral origin, synthetic waxes and the various fractions of waxes of natural origin, all of these waxes having the two characteristics (needle penetration, melting point) mentioned above.

The waxes (I) may especially be chosen from rice bran wax, carnauba wax, ouricury wax, candelilla wax, montan waxes, sugar cane waxes, and certain polyethylene waxes which satisfy the criteria for the waxes (I).

The composition may comprise an amount of the mixture of waxes I ranging from 10% to 30% by weight, relative to the total weight of the composition, preferably from 13% to 25% and better still at least 15%, and especially from 15% to 20%.

The wax I or the mixture of waxes I may comprise at least a first wax (Ia) having a melting point greater than or equal to 77° C. and less than 83° C. Such a wax may be, for example, rice bran wax.

The wax I or the mixture of waxes I may also comprise at least one second wax (Ib) having a melting point ranging from 83° C. to 110° C. This second wax (Ib) may be used alone in the mixture of waxes I or may be combined with the wax (Ia). Such a wax (Ib) may be, for example, carnauba wax, ouricury wax or montan waxes. Carnauba wax is preferably used.

The wax I or the mixture of waxes I may additionally comprise a third wax (Ic) having a melting point greater than or equal to 70° C. and less than 77° C. Such a wax may be, for example, candelilla wax.

The composition may comprise a mixture of waxes I containing at least one first wax (Ia) and at least one second wax (Ib) as defined previously.

Said mixture of waxes I may comprise from 35% to 65% by weight of wax (Ia), relative to the total weight of said mixture of waxes I, and from 65% to 35% by weight of wax (Ib).

Preferably, said mixture of waxes I may comprise, in addition to the first and second waxes (Ia, Ib), a third wax (Ic) as defined previously. This third wax may be present in the composition in a content ranging from 5% to 20% by weight, relative to the total weight of the mixture of waxes I.

Advantageously, the first, second and third waxes (Ia, Ib, Ic) may be present in the composition in a weight ratio (weight/total weight of waxes I) ranging, respectively, from:
wax (Ia): 0.35 to 0.5;
wax (Ib): 0.35 to 0.5;
wax (Ic): 0.05 to 0.2.

The composition may comprise, in addition to the mixture of wax I, at least one polymer system capable of forming a film that produces, at a concentration of 7% in water, a greater than 1%, preferably greater than 1.2% and better still greater than 1.5%, shrinkage of isolated stratum corneum, at 30° C. and under a relative humidity of 40%. This shrinkage is measured with a dermometer in accordance with the method described below. Such polymers give very good curling to the eyelashes.

The expression “polymer system” is understood to mean either a polymer alone or a polymer combined with at least one other polymer or a polymer combined with at least one plasticizer, so as to obtain the desired mechanical properties.

The expression “capable of forming a film” is understood to mean a polymer system that allows a film to be formed: when it is spread on glass, the polymer system must dry without cracking.

The film-forming polymer of the polymer system may be a polymer of natural origin or a synthetic polymer. The film-forming polymer may especially be a water-soluble or water-dispersible polymer.

The expression “polymer of natural origin” is understood to mean polymers of plant origin and of animal origin.

As polymers of plant origin, mention may especially be made of proteins and protein hydrolyzates, and more particularly extracts of cereals, of leguminous plants and of oleaginous plants, such as extracts of wheat, of corn, of rye, of Triticum aestivum wheat, of buckwheat, of sesame, of spelt, of pea, of bean, of lentil, of soybean and of lupin.

As polymers of animal origin, use may be made of polymers obtained from the carapace of insects or crustaceans. Mention may be made, for example, of chitin and its derivatives, in particular chitosan which is a deacetyl derivative of chitin, as well as chitosan derivatives such as hydroxypropyl chitosan, the succinyl derivative of chitosan, chitosan lactate, chitosan glutamate and carboxymethyl chitosan succinamide.

The polymers of natural origin, which are optionally modified, may be chosen, for example, from shellac resin, sandarac gum, dammar resins, elemi gums, copal resins and cellulose derivatives.

As suitable water-soluble polymers of natural origin, mention may be made of the hydroxypropyl chitosan sold under the name “HPCH powder” by Ichimaru Pharcos and the wheat protein hydrolyzate sold under the name “Tritisol” by the company Croda (having a molecular weight of about 250 000 daltons).

The synthetic polymers may be of polycondensate type or of radical type. As polycondensates, mention may be made of anionic, cationic, nonionic or amphoteric polyurethanes, polyurethane-acrylics, polyurethane-polyvinylpyrrolidones, polyester-polyurethanes, polyether-polyurethanes, polyurea-urethanes, polyureas, sulfopolyesters (which are described in particular in Patents U.S. Pat. No. 3,734,874, U.S. Pat. No. 4,233,196 and U.S. Pat. No. 4,304,901) and mixtures thereof.

As radical polymers, mention may be made of acrylic polymers, acrylic/styrene copolymers and vinyl copolymers such as vinyl ester copolymers.

As suitable synthetic polymer, mention may be made in particular of the polyester-polyurethane dispersions sold under the names “SANCURE 2060” (polyester-polyurethane), “SANCURE 815” (polyester-polyurethane) or else sulfopolyesters based on isophthalate/sulfoisophthalate, and more particularly the sulfopolyesters obtained by condensation of diethylene glycol, cyclohexanedimethanol, isophthalic acid and sulfoisophthalic acid, in particular those sold under the name AQ55S by Eastman.

When the film-forming polymer does not by itself allow a film having the characteristics mentioned previously to be obtained, it is possible to add a compound whose function is to modify the properties of the film-forming polymer in order to obtain the desired polymer system. Thus, said polymer system may comprise at least one auxiliary film-formation agent which makes it possible to obtain a film having the characteristics as described previously. The auxiliary film-formation agent makes it possible in particular to obtain a film which allows good curling of the eyelashes to be obtained.

In this case, the polymer system comprises a mixture of one or more film-forming polymers and at least one auxiliary film-formation agent.

Such an auxiliary film-formation agent may be chosen from any compound known to those skilled in the art as being capable of fulfilling the desired function, and may be chosen in particular from plasticizers. In addition, when the polymer system comprises at least one aqueous dispersion of particles of film-forming polymer, the auxiliary film-formation agent may also be chosen from coalescers. This auxiliary agent can be water-soluble or water-insoluble and may optionally be in the form of an aqueous dispersion.

In particular, mention may be made, alone or as a mixture, of the customary plasticizers or coalescers, such as:
glycols and their derivatives, such as diethylene glycol ethyl ether, diethylene glycol methyl ether, diethylene glycol butyl ether or else diethylene glycol hexyl ether, ethylene glycol ethyl ether, ethylene glycol butyl ether or ethylene glycol hexyl ether;
glycerol esters;
propylene glycol derivatives and in particular propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether;
acid esters, in particular carboxylic acid esters, such as citrates, phthalates, adipates, carbonates, tartrates, phosphates and sebacates;
oxyethylenated derivatives such as oxyethylenated oils, in particular plant oils such as castor oil; and silicone oils; and
water-soluble polymers having a low glass transition temperature of less than 25° C., preferably less than 15° C.

The amount of auxiliary film-formation agent may be chosen by a person skilled in the art on the basis of his or her general knowledge, so as to obtain a polymer system which leads to a film having the desired mechanical properties, while at the same time retaining acceptable cosmetic properties for the composition.

The composition may comprise from 0.1% to 10% by weight, preferably from 0.3% to 7%, of film-forming polymer solids relative to the total weight of the composition.

The polymer system used (polymer(s) or polymer and plasticizer) may be present in particular in an active material (A.M.) amount ranging from 0.1 to 15%, and better still from 0.3 to 10%, of the total weight of the composition.

In addition, the composition may comprise at least one wax II, referred to as soft wax, having a melting point greater than or equal to 50° C. and less than 70° C., and a needle penetration of greater than 7.5, and preferably less than or equal to 217, measured according to the conditions defined previously for the waxes I. This wax II makes it possible in particular to impart flexibility to the makeup deposited on the eyelashes.

These waxes II may be chosen in particular from beeswax, lanolin waxes, paraffin waxes, ceresin waxes, microcrystalline waxes, ozokerites, spermacetis, certain polyethylene waxes with a molecular weight such that they satisfy the criteria of the waxes II, and hydrogenated plant oils.

Among the hydrogenated plant oils, mention may be made of hydrogenated jojoba waxes and hydrogenated oils which are obtained by catalytic hydrogenation of fatty substances composed of a linear or non-linear C₈-C₃₂ fatty chain and which have the qualities corresponding to the definition of waxes. Mention may be made in particular of hydrogenated sunflower oil, hydrogenated castor oil, hydrogenated coconut oil and hydrogenated lanolin.

Advantageously, the waxes I and the waxes II are present in the composition in a waxes I/wax II weight ratio which can range from 2 to 5 and preferably from 2.5 to 3.5.

The composition may contain emulsifying surfactants present in a proportion ranging from 2 to 30% by weight relative to the total weight of the composition, and better still from 5% to 20%. These surfactants may be chosen from anionic or nonionic surfactants. Reference may be made to the document “Kirk-Othmer Encyclopedia of Chemical Technology”, Volume 22, p. 333-432, 3rd Edition, 1979, Wiley, for the definition of the properties and functions (emulsifying) of surfactants, in particular p. 347-377 of this reference, for the anionic and nonionic surfactants.

The surfactants preferably used in the compositions according to this example are:
among the nonionic surfactants: fatty acids, fatty alcohols, polyethoxylated or polyglycerolated fatty alcohols such as polyethoxylated stearyl or cetylstearyl alcohols, fatty acid esters of sucrose, alkylglucose esters, in particular polyoxyethylenated fatty esters of (C₁-C₆)alkylglucose;
among the anionic surfactants: C₁₆-C₃₀ fatty acids neutralized with amines, ammonia or alkali metal salts.

Surfactants which allow an oil-in-water emulsion to be obtained are preferably used.

In the composition, water may advantageously represent from 30 to 80% by weight of the total weight of the composition.

In addition, the composition may comprise at least one thickener, preferably of hydrophilic nature. This may be chosen, for example, from carboxyvinyl polymers (carbomer), acrylic copolymers such as acrylate/alkylacrylate copolymers, polyacrylamides, polysaccharides, natural gums and clays.

The composition may advantageously comprise a silicone surfactant having an HLB ranging from 8 to 16. Preferably, such a surfactant is a dimethicone copolyol. When the composition according to the invention is deposited on the eyelashes, the silicone surfactant allows better staying power over time and better resistance to the mechanical stresses of the composition thus deposited.

The dimethicone copolyols may be chosen from the compounds of the general formula (I):

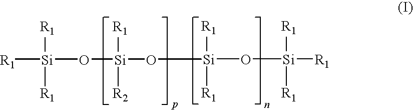

in which formula:
R₁, which may be identical or different, represent a hydrogen atom, a linear or branched C₁-C₃₀ alkyl radical or a phenyl radical;
R₂, which may be identical or different, represent —(CₓH₂ₓ)—(OC₂H₄)ₐ—(OC₃H₆)_{b}—OR₃,
R₃, which may be identical or different, are chosen from a hydrogen atom, a linear or branched alkyl radical having from 1 to 12 carbon atoms and a linear or branched acyl radical having from 2 to 12 carbon atoms;
n varies from 0 to 1000;
p varies from 1 to 30;
a varies from 0 to 50;
b varies from 0 to 50;
a+b is greater than or equal to 1;
x varies from 1 to 5;
the number-average molecular weight being greater than or equal to 15 000 and preferably between 25 000 and 75 000.

Preferably, oxyalkylenated silicones of general formula (I) which satisfy at least one, and preferably all, of the conditions below are used:
R₁ denotes a methyl radical;
R₃ represents a hydrogen atom, a methyl radical or an acetyl radical, and preferably hydrogen;
p varies from 8 to 20;
a is between 5 and 40 and preferably between 15 and 30;
b is between 5 and 40 and preferably between 15 and 30;
x is equal to 2 or 3; and
n varies from 20 to 600, preferably from 50 to 500 and more particularly still from 100 to 300.

Such silicones are described, for example, in patent U.S. Pat. No. 4,311,695, which is included by way of reference.

Dimethicone copolyols were presented in particular by Dow Corning during the 17th international congress of the I.F.S.C.C. of October 1992 and reported in the article “Water-soluble dimethicone copolyol waxes for personal care industry” by Linda Madore et al., pages 1 to 3.

These dimethicone copolyols are water-soluble polydimethylsiloxanes (PDMS) containing one or more ether functions (oxyalkylene, in particular oxyethylene and/or oxypropylene).

Such dimethicone copolyols are sold in particular by Goldschmidt under the name ABIL B8851 or ABIL B88183. Mention may also be made of the compounds KF 351 to 354 and KF 615 A sold by Shin Etsu or DMC 6038 from Wacker.

The dimethicone copolyol derivatives which can be used may be, in particular, dimethicone copolyols containing a phosphate, sulfate, myristamide propyldimethylammonium chloride, stearate, amine, glycomodified, etc. group. The compounds sold by Siltech under the name Silphos A100, Siltech amine 65, Silwax WDIS and myristamido silicone quat, or by Phoenix under the name Pecosil PS 100, may be used in particular as dimethicone copolyol derivatives.

The derivatives sold by Wacker under the name VP 1661, or by Dow Corning under the name 2501 cosmetic wax, may also be used. The derivatives sold by Wacker under the name VP 1661, or by Dow Corning under the name 2501 cosmetic wax, can also be used.

The silicones most particularly preferred are, for example, those sold by Dow Corning under the tradename Q2-5220 and by Rhone Poulenc under the name MIRASIL DMCO.

### EXAMPLE 1

A mascara having the following composition was prepared:

| rice bran wax | 7 | g |
|---|---|---|
| carnauba wax | 7 | g |
| candelilla wax | 2.8 | g |
| triethanolamine stearate | 8.4 | g |
| beeswax | 6 | g |
| wheat protein hydrolyzate sold under the name | 0.31 | g AM |
| “Tritisol” by Croda | | |
| hydroxyethyl cellulose | 1.5 | g |
| pigments | 8 | g |
| preservatives | qs | |
| water | qs for 100 | g |

The waxes, the surfactant and the preservatives were melted and mixed together at 90° C. The pigments were dispersed in the molten mixture at 90° C. The Tritisol and the hydroxyethyl cellulose were dissolved in the cold water. The aqueous phase was then heated to 90° C. and poured into the molten mixture with stirring, while maintaining the temperature at about 90° C. until the mixture was homogeneous. Next, the composition was cooled to ambient temperature.

A mascara was thus obtained in the form of a waxes-in-water dispersion in which the wax particles have a size greater than 1.5 μm.

### EXAMPLE 2

A mascara having the following composition is prepared:

| rice bran wax | 10 | g |
|---|---|---|
| carnauba wax | 8 | g |
| candelilla wax | 1 | g |
| triethanolamine stearate | 9 | g |
| beeswax | 4 | g |
| wheat protein hydrolyzate sold under the name | 0.4 | g AM |
| “Tritisol” by Croda | | |
| hydroxyethyl cellulose | 1.5 | g |
| pigments | 5 | g |
| preservatives | qs | |
| water | qs for 100 | g |
| dimethicone copolyol sold under the name | 0.3 | g |
| “Q2-5520” by Dow Corning | | |

### EXAMPLE 3

A mascara having the following composition is prepared:

| rice bran wax | 8 | g |
|---|---|---|
| carnauba wax | 8 | g |
| paraffin wax | 2 | g |
| triethanolamine stearate | 9 | g |
| beeswax | 3 | g |
| copolymer of diglycol/cyclohexane dimethanol/ | 1 | g |
| isophthalates/sulfoisophthalates sold under the name | | |
| “Eastman AQ-55S” by Eastman | | |
| hydroxyethyl cellulose | 1.2 | g |
| pigments | 7 | g |
| preservatives | qs | |
| water | qs for 100 | g |

### EXAMPLE 4

A mascara having the following composition was prepared:

| carnauba wax | 20.1 | g |
|---|---|---|
| polyoxyethylenated (30 EO) glyceryl monostearate | 6.71 | g |
| (TAGAT S from Goldschmidt) | | |
| hydroxyethyl cellulose | 1 | g |
| (Cellosize QP4400M from Amerchol) | | |
| gum arabic | 1.5 | g |
| panthenol | 1 | g |
| pigment | 5 | g |
| sodium hydroxide qs pH 7 | | |
| preservative | qs | |
| water | qs for 100 | g |

B—Other Examples of Compositions

The composition may also comprise at least one self-emulsifying wax and one polymer latex, in particular as described in Application EP 1 832 277.

The composition may thus comprise a polymer latex, a self-emulsifying wax in an amount of at least 15% by weight relative to the weight of the composition, and water. Such a composition may not comprise an additional emulsifier.

The expression “self-emulsifying” wax should be understood to mean a chemically modified wax that contains at least one emulsifier compound, for example a nonionic emulsifier.

Examples of self-emulsifying waxes are, for example, chosen from waxes of animal or plant, mineral or synthetic origin, for example beeswaxes, lanolin waxes and Chinese insect waxes, for example rice waxes, carnauba wax, candelilla wax and ouricury wax, cork fiber waxes, sugar cane fiber waxes, Japan waxes, sumach wax and cotton wax, paraffins, microcrystalline waxes, montan waxes and ozokerites, polyolefin waxes, for example polyethylene waxes, waxes obtained by Fischer-Tropsch synthesis, waxy copolymers and their esters, and silicone waxes and fluoro waxes.

Hydrogenated oils of animal or plant origin may be present in the self-emulsifying waxes. The self-emulsifying waxes may be obtained from numerous manufacturers and suppliers, for example PEG-20 sorbitan beeswaxes (Atlas G-1726, Uniqema; Nikkol GBW-125, Nikko), PEG-6 beeswax (ESTOL 375, Uniqema), PEG-8 beeswax (Apifil, Gattefosse), PEG-12 beeswax and PEG-12 carnauba wax

The latices used are aqueous dispersions of at least one polymer, for example a film-forming polymer chosen from copolymers of methacrylic acid and its esters, copolymers of methacrylate, acrylates which herein refer to a copolymer of at least two or more monomers chosen from the group consisting of acrylic acid, methacrylic acid, and their simple esters, for example the esters having a small number of carbon atoms such as methyl, ethyl esters and ethylhexyl esters. Such copolymers, which may be in the form of an aqueous dispersion, are commercially available from numerous suppliers and manufacturers, including Covacryl A15 and Covacryl E by Wackherr, Luviflex Soft from BASF, Luvimer 36D from BASF, Yodosol GH800 from National Starch and Carboset XL-40 from BF Goodrich.

In implementation examples, acrylate copolymers form an aqueous dispersion of the ethyl ester of acrylic acid and the methyl ester of methacrylic acid, which is commercially available from Daito Kasei under the tradename Daitosol 5000AD (CAS No. 2135-39-1, which is an aqueous emulsion having a solids content of about 50%).

Other methacrylate copolymers include acrylate copolymers (acrylates/ethylhexyl acrylate copolymer, sold by Daito Kasei under the tradename Daitosol 5000SJ), butyl acrylate/hydroxypropyl dimethicone acrylate copolymers (Granacrysil BAS by Grant Industries, Inc.), acrylates/C12-C22 alkylmethacrylate copolymers (Allianz OPT by ISP), isododecane and acrylates copolymers (Giovarez AC-5099M by Phoenix), and acrylates/octylacrylamide copolymers (Dermacryl-79 by National Starch & Chemical Company).

The polymer content of the latex (including the aqueous dispersion in which it may be sold) in the composition may range from 0.1 to 40%, for example between 0.5 and 30%, or even 5 to 20%, relative to the total weight of the composition.

The composition may also comprise a film-forming polymer, one, for example, of the silicone oils which may be used in the invention, including non-volatile linear polydimethylsiloxanes (PDMSs), which are liquid at ambient temperature, polydimethylsiloxanes which may be substituted by fluoro groups, groups such as hydroxyls, thiols or amines, aliphatic (e.g., alkyl) groups, or aromatic (e.g., phenyl) groups, which are attached and/or at the end of the chain of silicones, these groups each containing from 2 to 4 carbon atoms; phenylsilicones, for example phenyl trimethicones, phenyl dimethicones, trimethylpentaphenyltrisiloxane, tetramethyltetraphenyltrisiloxane, phenyltrimethylsiloxydiphenylsiloxanes (e.g., DC555 from Dow Corning), diphenyldimethicones, diphenylmethyldiphenyltrisiloxanes and 2-phenylethyltrimethylsiloxysilicates. Other silicone oils include polysiloxanes modified with fatty acids, alcohol acids or polyoxyalkylenes, fluorosilicates and perfluoro oils.

The composition is for example in accordance with one of examples 1 to 4 from Application EP 1 832 177 A1.

The self-emulsifying wax may be present in an amount between 20 and 30%, better still between 20 and 25%, of the total weight of the composition.

C—Other Examples of Compositions

The composition may also be produced in accordance with European patent Application EP 1 752 136 A2.

Such a composition comprises an aqueous phase, a fatty phase, a structuring agent, and a polyurethane/poly(meth)acrylate graft copolymer. This graft polymer is defined in paragraphs [0009] and [0010] of Application EP 1 752 136 A2.

Another composition example which may be suitable is a composition without the above graft copolymer.

Example 1 of Application EP 1 752 136 A2 is a composition example in which the copolymer is present in the form of a latex, the weight-average size of the particles being, for example, between 90 and 110 nm and the number-average size around 80 nm with a glass transition temperature T_{G} ranging from −60° C. to 100° C., the polymer being, for example, a poly(meth)acrylate comprising a polymethyl methacrylate.

The copolymer may be present in an amount ranging from 0.1% to 20% by weight relative to the total weight of the composition.

The structuring agent may comprise at least one wax, for example tetradecyl-octadecanyl-behenate or hexadecyl-cosanyl hexacosanate.

This wax may be present in an amount ranging from 0.1% to 40% by weight relative to the total weight of the composition.

The structuring agent may also comprise a non-waxy polymer, for example an ester-terminated polyamide given by the formula represented in claim 10 of Application EP 1 752 136 A2.

D—Other Examples of Compositions

The composition may also be as disclosed in Application EP 0 832 637 A1.

The composition may thus comprise:

silicone (VII) or a (VIII)).
(a) at least one aqueous phase comprising at least one film-forming polymer;
(b) at least one fatty phase comprising at least one wax; and
(c) at least one silicone agent chosen from:
   (i) oxyalkylenated silicones; and/or
   (ii) linear polysiloxane-polyoxyalkylene block copolymers.

These compositions are preferably present in the form of an emulsion of the fatty phase in the aqueous phase or a dispersion of the aqueous phase in the fatty phase.

The composition may therefore comprise a silicone agent which may be an oxyalkylenated silicone.

In particular, the oxyalkylenated silicones do not contain an alkyl or alkoxy group having 8 to 22 carbon atoms and linked directly to a silicon atom.

The oxyalkylenated silicones are chosen from the compounds of general formula (I) also known as dimethicone copolyols:

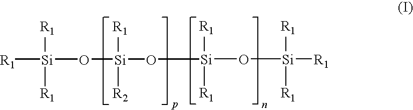

in which formula:
R₁, which may be identical or different, represent a hydrogen atom, a linear or branched C₁-C₃₀ alkyl radical or a phenyl radical;
R₂, which may be identical or different, represent —(CₓH₂ₓ)—(OC₂H₄)ₐ—(OC₃H₆)_{b}—OR₃,
R₃, which may be identical or different, are chosen from a hydrogen atom, a linear or branched alkyl radical having from 1 to 12 carbon atoms and a linear or branched acyl radical having from 2 to 12 carbon atoms;
n varies from 0 to 1000;
p varies from 1 to 30;
a varies from 0 to 50;
b varies from 0 to 50;
a+b is greater than or equal to 1;
x varies from 1 to 5;
the number-average molecular weight being greater than or equal to 15 000 and preferably between 25 000 and 75 000.

Preferably, oxyalkylenated silicones of general formula (I) which satisfy at least one, and preferably all, of the conditions below are used:
R₁ denotes a methyl radical;
R₃ represents a hydrogen atom, a methyl radical or an acetyl radical, and preferably hydrogen;
p varies from 8 to 20;
a is between 5 and 40 and preferably between 15 and 30;
b is between 5 and 40 and preferably between 15 and 30;
x is equal to 2 or 3; and
n varies from 20 to 600, preferably from 50 to 500 and more particularly still from 100 to 300.

Such silicones are described, for example, in patent U.S. Pat. No. 4,311,695, which is included by way of reference.

Dimethicone copolyols were presented in particular by Dow Corning during the 17th international congress of the I.F.S.C.C. of October 1992 and reported in the article “Water-soluble dimethicone copolyol waxes for personal care industry” by Linda Madore et al., pages 1 to 3.

These dimethicone copolyols are water-soluble polydimethylsiloxanes (PDMS) containing one or more ether functions (oxyalkylene, in particular oxyethylene and/or oxypropylene).

Such dimethicone copolyols are sold in particular by Goldschmidt under the name ABIL B8851 or ABIL B88183. Mention may also be made of the compounds KF 351 to 354 and KF 615 A sold by Shin Etsu or DMC 6038 from Wacker.

The dimethicone copolyol derivatives which can be used may be, in particular, dimethicone copolyols containing a phosphate, sulfate, myristamide propyldimethylammonium chloride, stearate, amine, glycomodified, etc. group. The compounds sold by Siltech under the name Silphos A100, Siltech amine 65, Silwax WDIS and myristamido silicone quat, or by Phoenix under the name Pecosil PS 100, may be used in particular as dimethicone copolyol derivatives.

The derivatives sold by Wacker under the name VP 1661, or by Dow Corning under the name 2501 cosmetic wax, may also be used. The derivatives sold by Wacker under the name VP 1661, or by Dow Corning under the name 2501 cosmetic wax, can also be used.

The silicones most particularly preferred are, for example, those sold by Dow Corning under the tradename Q2-5220 and by Rhone Poulenc under the name MIRASIL DMCO.

When linear polysiloxane-polyoxyalkylene block copolymers are used, these preferably have the general formula (II) below:

([Y(R2SiO)a′R′2SiYO][(Cn′H2n′O)b′])c′ (II)

in which:
R and R′, which are identical or different, represent a monovalent hydrocarbon radical containing no aliphatic unsaturation;
n′ is an integer between 2 and 4;
a′ is an integer greater than or equal to 5;
b′ is an integer greater than or equal to 4;
c′ is an integer greater than or equal to 4;
Y represents a divalent organic group which is attached to the adjacent silicon atom by a carbon-silicon bond and to a polyoxyalkylene block by an oxygen atom;
the average molecular weight of each siloxane block is between about 400 and about 10 000, that of each polyoxyalkylene block being between about 300 and about 10 000;
the siloxane blocks represent from about 10% to about 90% by weight of the block copolymer; and
the average molecular weight of the block copolymer being at least 3000.

The radicals R and R′ are more preferably chosen from the group comprising alkyl radicals such as, for example, methyl, ethyl, propyl, butyl, pentyl, hexyl, octyl, decyl and dodecyl radicals; aryl radicals such as, for example, phenyl and naphthyl radicals; aralkyl radicals such as, for example, benzyl and phenylethyl; tolyl, xylyl and cyclohexyl radicals.

The divalent radical Y is preferably —R″—, —R″—CO—, —R″—NHCO—, —R″—NH—CO—NH—R′″—NHCO or —R″—OCONH—R′″—NHCO—, where R″ is a divalent alkylene group such as, for example, ethylene, propylene or butylene and R′″ is a divalent alkylene group or a divalent arylene group such as —C₆H₄—, —C₆H₄—C₆H₄—, —C₆H₄—CH₂—C₆H₄— or —C₆H₄—CH(CH₃)₂—C₆H₄—.

Even more preferably, Y represents a divalent alkylene radical and more particularly the —CH₂—CH₂—CH₂— radical.

The preparation of the block copolymers used in the context of the present invention is described in particular in European Application EP 0 492 657 A1.

According to one particular embodiment of the invention, the block copolymer is chosen from the following copolymers:

[[(CH₃)₂SiO]₄₁(CH₃)₂SiCH₂CH(CH₃)CH₂—O(C₂H₄O)₁₈—(C₃H₆O)₃₃CH₂CH(CH₃)CH₂]_{16.1}

[[(CH₃)₂SiO]₃₁(CH₃)₂SiCH₂CH(CH₃)CH₂—O(C₂H₄O)₂₀—(C₃H₆O)₂₉CH₂CH(CH₃)CH₂]_{13.3}

[[(CH₃)₂SiO]₉(CH₃)₂SiCH₂CH(CH₃)CH₂—O(C₂H₄O)₂₀—(C₃H₆O)₂₉CH₂CH(CH₃)CH₂]_{26.3}

[[(CH₃)₂SiO]₁₆(CH₃)₂SiCH₂CH(CH₃)CH₂—O(C₂H₄O)₁₈—(C₃H₆O)₂₀CH₂CH(CH₃)CH₂]_{21.5}

[[(CH₃)₂SiO]₉(CH₃)₂SiCH₂CH(CH₃)CH₂—O(C₂H₄O)₅—CH₂CH(CH₃)CH₂]_{4.8}

The silicone agents used in the compositions may be water-soluble or liposoluble. Depending on their water solubility or liposolubility, they are introduced, respectively, into the aqueous phase or the fatty phase.

The waxes that can be used in the composition according to these examples possess, as a general rule, a melting point between 40 and 110° C. and have a needle penetration, at 25° C., between 3 and 40, as measured according to US standard ASTM D 5 or according to French standard NFT 004. The principle for measuring the penetration of a needle according to the ASTM D 5 and AFT 004 standards comprises measuring the depth, expressed in tenths of a millimeter, to which a standardized needle which weighs 2.5 g penetrates when placed in a needle holder weighing 47.5 g, i.e. a total of 50 g, the needle being placed on the wax for 5 seconds.

The waxes that can be used according to this composition example may be chosen from animal waxes, plant waxes, mineral waxes, synthetic waxes and various fractions of natural waxes, all of the waxes having the two physical characteristics mentioned previously.

Among the animal waxes, mention may be made of beeswaxes, lanolin waxes and Chinese insect waxes.

Among the plant waxes, mention may be made of rice waxes, carnauba waxes, candelilla waxes, ouricury waxes, cork fibre waxes, sugar cane waxes, Japan waxes, sumach wax and cotton wax.

Among the mineral waxes, mention may be made of paraffins, microcrystalline waxes, montan waxes and ozokerites.

Among the synthetic waxes, use may be made, in particular, of polyethylene waxes, waxes obtained by Fischer-Tropsch synthesis, waxy copolymers and esters thereof, and silicone waxes.

It is also possible to use hydrogenated animal or plant oils which still satisfy the two physical characteristics mentioned previously.

Among these oils, mention may be made of hydrogenated jojoba waxes and hydrogenated oils which are obtained by catalytic hydrogenation of fatty substances composed of a linear or non-linear C₈-C₃₂ fatty chain, hydrogenated sunflower oil, hydrogenated castor oil, hydrogenated coconut oil, hydrogenated lanolin and hydrogenated palm oils.

The waxes that can be used are preferably solid and rigid at a temperature below 50° C.

These waxes may be in the form of stable dispersions of colloidal particles of wax as may be prepared according to known methods, such as “Microemulsions Theory and Practice”, L. M. Prince Ed., Academic Press (1977), pages 21-32.

The composition may comprise from 6 to 40%, preferably from 10 to 25%, of wax by weight relative to the total weight of the composition.

The composition may also comprise at least one film-forming polymer, in solution or in dispersion in the aqueous phase.

The composition may comprise an amount of film-forming polymer ranging from 0.1 to 25% by weight relative to the total weight of the composition. Advantageously, the compositions according to this example comprise an amount ranging from 1 to 10%, by weight relative to the total weight of the composition, of film-forming polymer.

The film-forming polymer may be chosen from:
keratin derivatives, such as keratin hydrolyzates and sulfonic keratins;
anionic, cationic, amphoteric or nonionic chitin or chitosan derivatives;
cellulose derivatives such as hydroxyethyl cellulose, hydroxypropyl cellulose, methyl cellulose, ethyl hydroxyethyl cellulose and carboxymethyl cellulose, and also quaternized derivatives of cellulose;
acrylic polymers or copolymers, such as polyacrylates or polymethacrylates;
polyvinylpyrrolidones and vinyl copolymers, such as copolymers of methyl vinyl ether and of malic anhydride or the copolymer of vinyl acetate and of crotonic acid;
water-dispersable anionic polyester and/or polyesteramide polymers comprising monomers bearing a function: —SO₃M, with M representing a hydrogen atom, an ammonium ion NH₄⁺ or a metal ion, such as for example an Na⁺, Li⁺, K⁺, Mg²⁺, Ca²⁺, Cu²⁺, Fe²⁺ or Fe³⁺ ion. Mention may be made, in particular, of the polymers described in documents U.S. Pat. No. 3,734,874; U.S. Pat. No. 4,233,196; and U.S. Pat. No. 4,304,901. Advantageously, film-forming polyester polymers based on at least one dicarboxylic acid, on at least one diol and at least one difunctional aromatic monomer also bearing an —SO₃M group as described above are chosen.
fatty chain polyesters, polyamides, and epoxy ester resins;
polyurethane polymers, especially anionic, cationic, nonionic or amphoteric polyurethanes, polyurethane-acrylics, polyurethane-polyvinylpyrrolidones, polyester-polyurethanes, polyether-polyurethanes, polyureas, polyurea-polyurethanes, and blends thereof;
polymers of natural origin, which are optionally modified, such as:
   gum arabics, guar gum, xanthan derivatives, karaya gum;
   alginates and carrageenates;
   glycoaminoglycans, hyaluronic acid and derivatives thereof;
   shellac resin, sandarac gum, dammar resins, elemi gums and copal resins.

The composition according to these examples D may comprise from 0.01 to 5% of silicone agent, more preferably still from 0.4 to 1.5% by weight relative to the total weight of the composition, especially from 10 to 30% by weight of silicone agent relative to the total weight of the film-forming polymer.

The components of the oily and aqueous phases may be independently dissolved or melted at a temperature of 85° C. and then mixed together.

The composition may be in the form of an oil-in-water emulsion or a water-in-wax dispersion.

When it is used in the form of an oil-in-water emulsion, the composition may contain emulsifying surfactants present in a proportion of between 2 and 30% by weight relative to the total weight of the composition. These surfactants may be chosen from anionic or nonionic surfactants. Reference may be made to the document “Kirk-Othmer Encyclopedia of Chemical Technology” Volume 22, p. 333-432, 3rd edition, 1979, Wiley, for the definition of the properties and functions (emulsifying) of the surfactants, in particular p. 347-377 of this reference, for the anionic and nonionic surfactants.

The surfactants preferably used in the compositions according to the invention are:
among the nonionic surfactants: fatty acids, fatty alcohols, polyethoxylated or polyglycerolated fatty alcohols such as polyethoxylated stearyl or cetylstearyl alcohols, fatty acid esters of sucrose, alkyl glucose esters, in particular polyoxyethylenated fatty esters of C₁-C₆ alkyl glucose;
among the anionic surfactants: amine stearates.

When the composition is in the form of an oil-in-water emulsion, the water advantageously represents from 30 to 80% by weight of the total weight of the composition.

When the composition is in the form of a water-in-wax dispersion, it also preferably comprises at least one thickener and at least one volatile solvent or volatile organic oil which will evaporate on contact with the skin or the eyelashes, but whose presence in the cosmetic composition is useful since they facilitate the spreading of the composition when it is applied to the skin or the eyelashes. Such spreading agents, referred to here as volatile solvents or oils, are generally organic compounds having a saturation vapor pressure at 25° C. at least equal to 0.5 millibar (i.e. 0.5×10² Pa).

The thickener may be chosen from organically modified clays, such as montmorillonites and hectorite derivatives, for example bentonite.

The concentration of volatile solvent or of volatile organic oil is between 35 and 70% by weight relative to the total weight of the composition. The volatile solvent or volatile organic oil may be chosen from isoparaffin, turpentine, isopropyl alcohol, ethyl alcohol, white spirit, silicone oils, such as hexamethyldisiloxane, cyclopentadimethylsiloxane or cyclotetramethylsiloxane, fluoro oils, such as those sold under the name Galden (Montefluos), or isoparaffin oils, such as those which are sold under the name ISOPAR (E, G, L or H) and also isododecane.

In this embodiment, the aqueous phase preferably represents from 0.1 to 25% by weight relative to the total weight of the composition.

Reference may be made to the document WO 91/12793 for the preparation of compositions in the form of a water-in-wax dispersion, these dispersions additionally having the property of being water-resistant.

The composition may additionally comprise at least one filler, such as:
talc, which is a hydrated magnesium silicate used in the form of particles generally of less than 40 microns;
micas, which are aluminosilicates of various compositions that are in the form of flakes having dimensions of 2 to 200 microns, preferably of 5 to 70 microns, and a thickness of between 0.1 to 5 microns, preferably from 0.2 to 3 microns, it being possible for these micas to be of natural origin, such as muscovite, margarite, roscoelite, lipidolite or biotite, or of synthetic origin;
starch, in particular rice starch,
kaolin, which is a hydrated aluminum silicate which is in the form of particles of isotropic shape having dimensions generally of less than 30 microns;
zinc and titanium oxides generally used in the form of particles having dimensions not exceeding a few microns;
calcium carbonate, magnesium carbonate or magnesium hydrogen carbonate;
microcrystalline cellulose;
silica;
powders of synthetic polymers, such as polyethylene, polyesters (polyethylene isophthalate or terephthalate) or polyamides, such as those sold under the tradename “Nylon” or “Teflon”, and silicone powders.

In the example below, all the percentages are given by weight of active material.

### EXAMPLE 1

| | |
|---|---|
| Beeswax | 3% |
| Carnauba wax | 3% |
| Paraffin | 13% |
| Black iron oxide | 7% |
| Triethanolamine stearate | 9% |
| Gum arabic | 3.5% |
| Hydroxyethyl cellulose | 1% |
| Oxyethylenated polydimethylsiloxane (*) | 0.6% |
| Poly(sodium methacrylate) | 0.25% |
| Preservatives | qs |
| Water | qs for |

(*) sold by Dow Corning under the commercial reference Q2 5220

The invention will be better understood on reading the following detailed description of non-limiting implementation examples of the latter and on examining the appended drawing, in which:

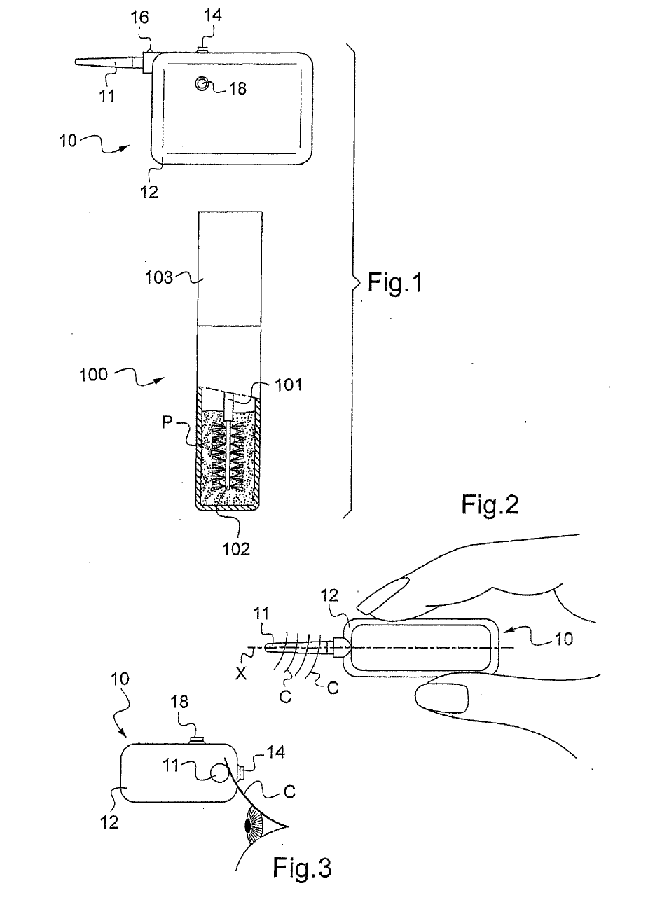

FIG. 1 illustrates an example of an assembly for the implementation of the invention;

FIGS. 2 and 3 illustrate the use of the finishing device to curl the eyelashes;

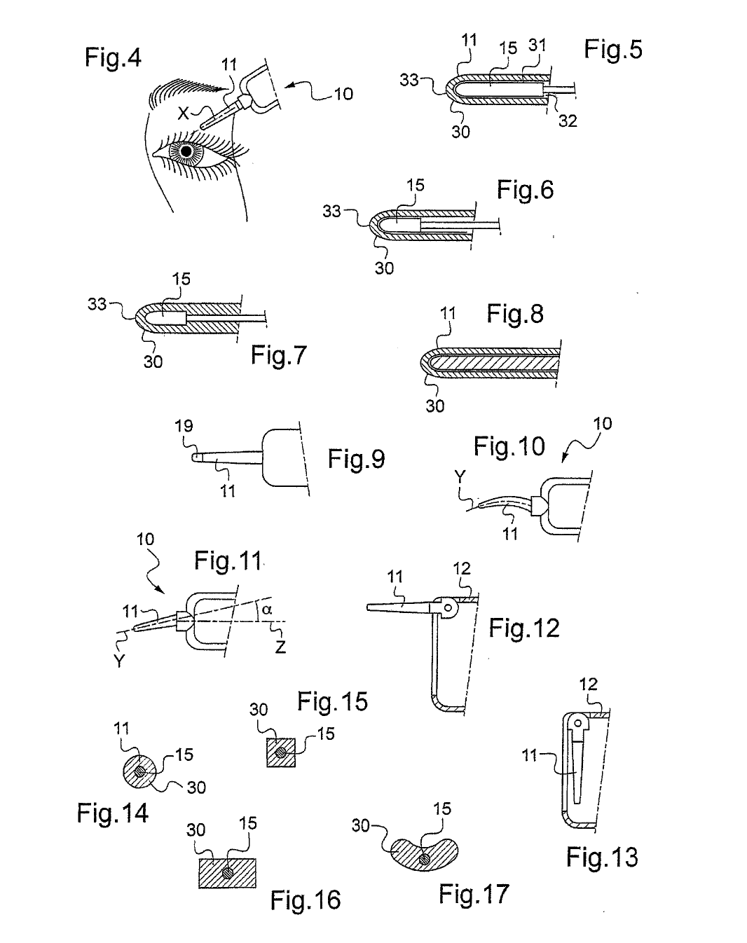

FIG. 4 illustrates the use of the finishing device to separate the eyelashes;

FIGS. 5 to 8 illustrate various examples for mounting a heating resistor in the heating support;

FIGS. 9 to 11 represent variants of the heating support;

FIGS. 12 and 13 illustrate the possibility of mounting the heating support in an articulated manner on the casing;

FIGS. 14 to 19 are schematic cross sections of variants of the heating support;

FIGS. 20 and 21 represent other variants of the heating support;

FIG. 22 represents a variant of the finishing device;

FIG. 23 represents the device from FIG. 22 after opening the casing;

FIG. 24 represents a variant of the finishing device;

FIG. 25 represents the device from FIG. 24, from the side;

FIG. 26 represents another variant of the finishing device; and

FIG. 27 illustrates the use of a finishing device with a vibrating source borne by a finger of the user.

FIG. 1 represents an assembly for the implementation of the invention, comprising, on the one hand, a finishing device 10 and, on the other hand, a packaging and application device 100 containing a composition to be applied to the eyelashes, which is for example any one of those described previously under A) to D).

Such a composition P is, for example, in a liquid, pasty or gelled form, and contains at least one volatile solvent, which is for example water or an organic solvent. This solvent, by evaporating, allows the composition to harden, for example by crosslinking or by coagulating.

The applicator comprises, for example, a wand 101 which is equipped, at one end with the application member 102 and at the other end with a gripping member 103 which also acts as a closure cap for the container and which is for example equipped so as to be screwed on the threaded neck of the container. The application member 102 is for example a brush or a comb. In the case of a brush, this may be injection molded or comprise a twisted core with bristles held between the turns of the core.

The container is for example conventionally equipped with a wiping member that makes it possible to wipe the wand 101 of the applicator and the application member 102 when removed from the container.

The finishing device 10 comprises a heating support 11 and a casing 12 that houses an electrical source, which is for example constituted by a flat 9 V battery. In one variant that is not represented, the electrical source is rechargeable and may be recharged for example by placing the application device in a stand. In another variant that is not represented, the 9 V battery is replaced by at least one 1.5 V cylindrical battery, or even two 1.5 V batteries placed side by side, or a 3 V button battery. The application device may also be powered by the mains.

The finishing device 10 may comprise a member 14 for controlling the operation of the heating support 11, that is for example in the form of a contactor, for example a push button, that makes it possible, when pushed in by the user, to electrically power a heating resistor 15, which is not visible in FIG. 1 but which is represented in FIG. 5 in particular.

The finishing device 10 may optionally comprise, as illustrated, a light 16 to signal to the user that the heating resistor 15 is switched on.

The heating resistor 15 may be chosen so that even powered continuously, the temperature at the surface of the heating support 11 remains, in an environment close to 25° C., below a temperature capable of leading to a risk of burning the user in case of accidental contact, for example a temperature of 90° C.

As a variant, for example in order to better control the temperature of the heating support 11, the finishing device 10 may comprise a temperature control member, for example in the form of a bimetal thermostat or an electronic control circuit, comprising for example a temperature probe integrated into the heating support 11.

Preferably, during operation, the surface temperature of the heating support 11 remains between 45° C. and 90° C., better still between 55 and 65° C., but it would not be outside the scope of the present invention if the temperature during operation is different, especially higher.

The surface temperature may vary by less than 5° C. when moving over the circumference of the heating support at a point on its length.

The heating support 11 may be produced in various ways and comprises for example an envelope 30 that defines a housing 31 intended to receive the heating resistor 15, open at one end 32, the distal end 33 of the envelope 30 having, for example, a rounded shape, as illustrated in FIG. 5. The housing 31 is, for example, central.

The outer surface of the envelope 30 may have a taper of around 3 to 5° for example.

The thickness of the envelope 30 is for example between 0.3 and 1.5 mm, being for example constant in the circumferential direction around the heating resistor.

The housing 31 is for example of substantially cylindrical shape, having a diameter of around 1.2 mm for example.

The heating resistor 15 has, for example, a diameter of 1 mm.

The length of the heating support 11 is, for example, between 2 and 40 mm, being for example around 30 mm.

The envelope 30 is, for example, made of metal, especially of aluminum or any other metal or alloy that is a good conductor of heat.

The envelope 30 may also be made from a filled synthetic material, for example a thermoplastic or crosslinked material filled with particles, especially aluminum, copper or magnesia particles.

The heating support 11 may be fastened relative to the casing 12. As a variant, as illustrated in FIGS. 12 and 13, the heating support 11 may be movable relative to the casing 12, for example between a usage position represented in FIG. 12 and a retracted position represented in FIG. 13.

The displacement of the heating support 11 may take place independently of its power supply or not. As a variant, the supply of power may only take place in the extended position of the heating support 11, whatever the manner in which the finishing device is produced.

Where appropriate, means for locking the heating support 11 in the retracted or extended position may be provided, these locking means being, for example, in the form of a relief that generates a sticking point in the movement during displacement of the heating support 11.

In the example from FIGS. 12 and 13, the heating support can be moved by around a quarter of a turn between the retracted position and the drawn-back position.

The passage from the retracted position to the drawn-back position may be carried out, where appropriate, against the return action of an elastic member, and the finishing device may comprise a member for unlocking the heating support 11, so that this automatically goes back to the extended position under the return action of the elastic member.

The heating support 11 may be produced with various shapes and orientations. In the example from FIG. 10, the possibility for the heating support 11 to extend along a curvilinear longitudinal axis Y is illustrated, the axis Y being, for example, substantially arc-shaped, for example having a curvature adapted to that of the fringe of eyelashes.

In the variant illustrated in FIG. 11, the longitudinal axis Y of the heating support 11 is rectilinear and extends by forming an angle a with the longitudinal axis Z of the casing 12 of the finishing device.

The application device 10 may comprise a cap, not represented, which may cover the heating support 11 before and after use. Such a cap may, where appropriate, make it easier to raise the temperature of the heating support owing to the confinement of the hot air.

It is possible to produce the heating support 11 with various shapes and especially various cross sections, especially on the portion intended to contact the eyelashes.

By way of example, various options for cross sections have been represented in FIGS. 14 to 19.

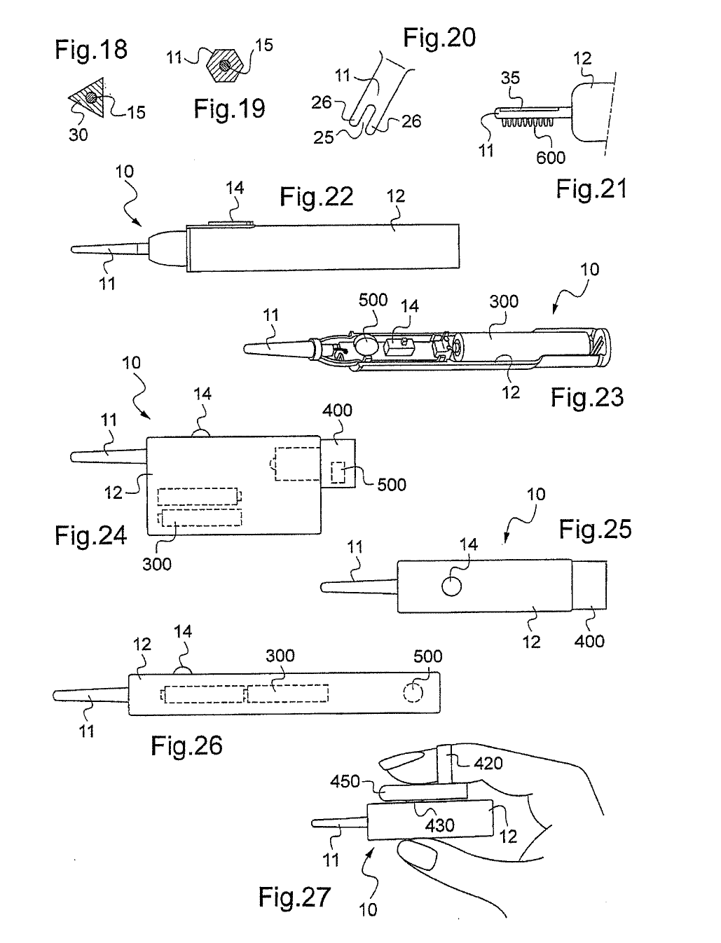

The heating support 11 may possess an envelope 30 that has an outer surface with a shape that is circular in cross section, as illustrated in FIG. 14, polygonal, for example triangular as illustrated in FIG. 18, hexagonal as illustrated in FIG. 19, square as illustrated in FIG. 15 or rectangular as represented in FIG. 16, with, where appropriate, at least one concave face as illustrated in FIG. 17.

The envelope 30 of the heating support 11 may have a thickness that varies depending on the position along the longitudinal axis Y of the heating support.

The variation in the thickness of the wall of the envelope 30 may be chosen as a function of the temperature to be achieved on the surface, as a function for example of the nature and of the position of the heating resistor 15 placed inside the envelope 30.

The envelope may also have a substantially constant wall thickness over most of the length of the heating support 11.

The heating resistor 15 may extend all along the part of the heating support 11 covered by the applicator tip 20, as illustrated in FIG. 5. As a variant, the heating resistor 15 may extend only over one part of this length, as illustrated in FIG. 6, being for example located at the end of the heating support 11.

The heating resistor 15 is for example inserted into a housing of the envelope 30, with a small clearance, so as to improve the heat transfer.

As a variant, the envelope 30 is overmolded over the heating resistor 15, as illustrated in FIG. 7. In this case it is possible to use a synthetic material such as silicone, for example filled with metallic or magnesia particles, in order to produce the envelope 30. The envelope may also be made of PTFE (Teflon®) or covered with PTFE.

The envelope 30 may be rigid or flexible. In one variant, the envelope 30 is made of glass.

In the example from FIG. 9, the heating support 11 comprises an end portion 19 that is made from a material that is a poor conductor of heat and/or which is not heated.

Represented in FIG. 20 is a variant of the heating support 11 with a recess 25 at the end, separating, for example, two tips 26. The latter may be used to separate the eyelashes, where appropriate.

In the example from FIG. 21, the hot surface 35 extends only over one portion of the heating support 11.

The hot surface 35 extends, for example, over an upper portion only of the heating support 11, as illustrated.

Except for the hot surface 35, the heating support is for example made from a material that is a poor conductor of heat, for example a plastic material.

The heating support 11 may optionally comprise, as illustrated by dotted lines in FIG. 21, at least one row of teeth away from the hot surface, for example on the opposite side to this surface.

Illustrated in FIGS. 22 and 23 is the possibility of the casing 12 having an elongated pen shape.

In FIG. 23 the possibility of the electrical source 300 comprising a cylindrical battery is illustrated.

The finishing device 10 may comprise a vibrating source.

In the examples from FIGS. 1 and 22, the vibrating source is integrated into the finishing device 10.

In the example from FIG. 1, its operation may be controlled by means of a switch 18, for example located on the casing 12 on a different face to that bearing the switch 14, so as to enable the user to act on the switches 14 and 18 with two digits of one hand.

In the example from FIGS. 22 and 23, it is, for example, the switch 14 that controls both the power supply for the heating support and for the vibrating source.

The vibrating source is for example an electric motor that rotates an off-center flyweight, the rotational speed of the motor being a function of the frequency of the vibrations to be produced, this rotational speed being, for example, between 4500 and 12 000 rpm.

The frequency of the vibrations is, for example, between 1 and 500 Hz, better still 50 to 200 Hz.

The motor 500 is for example a motor contained in a disk-shaped casing, as can be seen in FIG. 23, and the orientation of the axis of rotation of the motor governs that of the vibrations transmitted to the heating support 11.

The heating support 11 is for example subjected to vibrations that are oriented longitudinally, that is to say parallel to its longitudinal axis or transversely, especially perpendicular to its longitudinal axis.

Represented in FIG. 24 is a finishing device 10 equipped in order to allow this device to be fastened to a removable vibrating source 400. The latter is, for example, engaged in a housing of the casing 12 in order to transmit the vibrations that is generates to the heating support 11.

The activation of the vibrations may be carried out, for example, by means of a contactor, present for example on the vibrating source 400, which allows the user to choose the operating mode of the device, namely heating with vibrations, vibrations alone or heating alone.

The finishing device 10 may be in an elongated form, as illustrated in FIG. 25, with, for example, a power supply comprising two cylindrical batteries placed end to end.

The removable vibrating source 400 may be positioned at the rear of the device.

Application WO 2006/090343, the content of which is incorporated by reference, discloses examples of removable vibrating sources.

In the embodiment variant from FIG. 26, the vibrating source is positioned at the rear of the device, which has a pen shape, and is not removable.

Illustrated in FIG. 27 is the possibility of making the finishing device 10 vibrate by means of a vibrating source 450 which is temporarily brought into contact with the device 10, for example by being pressed against the casing 12.

The vibrating source 450 is then present, for example, in the form of a ring which is equipped with a means 420 for mounting on the finger of the user.

The vibrating source 450 may comprise a contactor 430 which is actuated when the ring is applied against the object to which it must transmit vibrations.

In the example illustrated, the contactor 430 is present on the face of the ring which is opposite the mounting means 420, and the user may transmit the vibrations to the heating support 11 by clasping the finishing device 10 between two digits, the vibrating ring being mounted on one of these digits.

The vibrating source may be produced other than with a motor that drives an off-center flyweight, for example comprising a motor that drives an eccentric, a piezoelectric vibrator or an electromechanical vibrator. The vibrating source may comprise a motor that drives a toothed wheel and a contact constituted for example by a resilient blade applied to this toothed wheel.

In order to implement the method according to the invention, the user applies the composition P to the eyelashes using the application member 102 in a conventional manner then, before the composition has finished drying, brings the heating support 11 into contact with the eyelashes by optionally lifting them, as illustrated in FIGS. 2 and 3.

In implementation variants of the invention, the finishing device comprises at least one row of teeth on the opposite side to the heating support.

The vibrating source, when it is added to the finishing device, for example added to it in a removable manner, may comprise teeth, which may be used to comb the eyelashes, for example.

The expression “comprising a” should be understood as being synonymous with “comprising at least one”, unless otherwise specified.

1. A method for making-up the eyelashes, comprising the steps made up of:
1) cold application of a composition (P) to the eyelashes using a non-heating applicator, this composition tending to set by drying;
2) before the composition dries completely, bringing a finishing device (10) comprising a hot surface that is subjected to vibrations into contact with the eyelashes thus coated with the composition, so as to accelerate the drying of the composition.

2. The method as claimed in claim 1, the application of the composition to the eyelashes being carried out in step 1) using a brush or a comb (102).

3. The method as claimed in either of the preceding claims, the hot surface being smooth.

4. The method as claimed in any one of the preceding claims, the hot surface being of tapered shape.

5. The method as claimed in one of the preceding claims, the hot surface having an axisymmetric shape, especially a symmetrical axisymmetric shape.

6. The method as claimed in any one of the preceding claims, the hot surface having a temperature less than or equal to 95° C., better still less than or equal to 90° C., for example between 35 and 85° C., even better still between 45° and 80° C., and preferably a homogeneous temperature.

7. The method as claimed in any one of the preceding claims, the hot surface being at least partially defined by a finger-shaped heating support.

8. The method as claimed in any one of the preceding claims, the hot surface being subjected to vibrations oriented parallel or transverse to the longitudinal axis of the hot surface.

9. The method as claimed in any one of the preceding claims, the hot surface being vibrated by means of a vibrating source (500) integrated into the finishing device.

10. The method as claimed in any one of claims 1 to 8, the hot surface being vibrated by means of a vibrating source (400) removably added to the finishing device or by means of a vibrating source (450) borne by a finger of the user and brought into contact with the finishing device.

11. The method as claimed in any one of claims 1 to 10, the hot surface being non-porous.

12. The method as claimed in any one of claims 1 to 11, the hot surface being free of composition (P) before its first contact with the eyelashes (C).

13. The method as claimed in any one of the preceding claims, the composition comprising one or more volatile solvents.

14. The method as claimed in any one of the preceding claims, the composition comprising at least one film-forming polymer.

15. The method as claimed in any one of the preceding claims, the composition comprising a latex.

16. The method as claimed in any one of the preceding claims, the composition comprising at least 30% water.

17. The method as claimed in any one of the preceding claims, the composition comprising at least one self-emulsifying wax.

18. The method as claimed in any one of the preceding claims, the composition comprising at least one hard wax.

19. An assembly comprising, within one and the same packaging:
a finishing device (10) comprising a heating surface;
a vibrating source that enables the heating surface to be vibrated, this vibrating source being integrated into the finishing device or possibly being removably fastened to this device or possibly being brought into contact with this device;
a composition (P) comprising at least one volatile solvent and possibly setting by drying.

20. The assembly as claimed in claim 19, the finishing device (10) comprising a finger-shaped heating support that at least partially defines the heating surface.

21. The assembly as claimed in claim 20, the heating support comprising an unheated portion at its free end.

22. The assembly as claimed in one of claims 19 to 21, the finishing device comprising at least one row of teeth.
